(19) United States
(12) Patent Application Publication
(10) Pub. No.: US 2026/0007809 A1
(43) Pub. Date: Jan. 8, 2026

(54) PREDONATION BAG SYSTEM CONTAINING AN ANTICOAGULANT, AND BLOOD BAG SYSTEM COMPRISING THE PREDONATION BAG SYSTEM

(71) Applicant: Blutspendedienst der Landesverbände des Deutschen Roten Kreuzes Niedersachsen, Sachsen-Anhalt,, Springe (DE)

(72) Inventors: Jana Schilling, Iianover (DE); Marcus Lach, Emmerthal (DE)

(21) Appl. No.: 18/870,369

(22) PCT Filed: Jun. 2, 2023
(86) PCT No.: PCT/DE2023/100420

§ 371 Date: Nov. 27, 2024

(30) Foreign Application Priority Data
Jun. 3, 2022 (DE) 10 2022 114 101.2

(51) Int. Cl. A61M 1/02

(52) U.S. Cl. CPC A61M 1/0272; A61M 2202/06; A61M 2205/0238

(57) ABSTRACT

The invention relates to a predonation bag system comprising a predonation bag to be filled with whole blood during the blood removal. an anticoagulant being added to the whole blood in the predonation bag and sample containers being automatedly filled with the whole blood from the predonation bag only in the laboratory for blood analysis. The predonation bag system is part of a blood bag system. The invention also relates to corresponding method for removing blood by means of the predonation bag system and for analyzing the whole blood of each donor.

The invention relates to a predonation bag system comprising a predonation bag for filling with whole blood during blood collection, wherein an anticoagulant is added to the whole blood in the predonation bag, so that the filling of the whole blood from the predonation bag into sample containers for blood analysis can only take place in the laboratory and not at the site of the blood donation. The predonation bag system is part of a blood bag system. Furthermore, a corresponding method for blood collection using the predonation bag system and for analyzing the whole blood of each donor is proposed.

### INTRODUCTION AND STATE OF THE ART

Blood products are stored by hospitals, blood banks and other medical facilities for use in blood transfusions. Different blood products are obtained from a donor's whole blood donation in order to administer the blood components a patient needs. On the one hand, the separation greatly reduces the risk of side effects and incompatibilities and, on the other hand, the separation of the blood into its components means that optimum conditions can be achieved for each of these components in terms of shelf life, storability and effectiveness.

As part of a blood donation, whole blood is first collected from individual donors. When donating blood, the blood of each donor, the so-called whole blood, is fed into a closed blood bag system. The blood bag system is designed in such a way that the whole blood is first collected in the whole blood bag (often also referred to as a collection bag) and then undergoes processing or separation treatments and the individual blood components obtained in this way are transferred into separate, connected bags (transfer bags). The blood from a donor is separated into its components. The separation is carried out by centrifugation. During centrifugation, an upper plasma layer, possibly a thinner intermediate layer known as the “buffy coat layer”, which contains the leukocytes and thrombocytes, and a lower layer of erythrocytes (red blood cells) are obtained.

It is necessary to separate the leukocytes. This is done by filtration before or after the separation step by centrifugation. Furthermore, the whole blood or the individual blood products can be subjected to a virus inactivation step. In many applications (such as for the present invention), a blood bag system is designed in such a way that a blood bag system can be assigned to exactly one donor only.

The above method requires careful labeling of the sample containers so that they can be clearly assigned to a donor and the corresponding blood bag system.

A large number of different blood bag systems are known for collecting, separating and storing blood components. As a rule, a closed sterile blood bag system, such as the Fenwal type, is used to collect whole blood and separate it into the blood components. This system contains a relatively large whole blood bag for collecting a blood donation, a filter and one or more transfer bags, which are connected to the whole blood bag by connecting tubes.

The different blood bag systems generally have in common that the whole blood flows from the donor into the whole blood bag without the use of external pumps or similar devices. To this end, blood collection involves inserting a vein access device, e.g. a venipuncture needle, into the donor's vein and withdrawing blood from the donor through the vein access device. The needle is connected to a tube via an adapter, through which the whole blood enters the whole blood bag. The venous access device, such as a venipuncture needle, tubing and bag form the blood bag system, which is pre-sterilized and disposed of after a single use.

The whole blood bag contains an anticoagulant, as blood begins to clot after some time and to adhere to the walls of the plastic surfaces with which it comes into contact or otherwise coagulate. The anticoagulants used must be registered in the European Pharmacopoeia. They are typically CPD (citrate-phosphate-dextrose) or ACD (acid-citrate-dextrose).

An important consideration for any blood collection technique or blood bag system is to ensure that the blood bag system is not contaminated by airborne bacteria or other foreign substances that could compromise the sterility of the system. Thus, the sterility of the prior art blood bag system described above is ensured by not exposing the flow paths and the interior of the bags to the external environment, or by keeping this exposure to a minimum. Such systems are generally referred to as “closed systems”.

Each time blood is donated, a donor's whole blood is tested for blood type and the presence of pathogens such as viruses and bacteria in the donor's blood. Typically, testing the collected blood requires the collection of one or more samples per donor at or near the time of collection. However, taking samples from the whole blood bag while exposing the collected blood in the whole blood bag to the external environment is undesirable. The anticoagulant in the whole blood bag also frequently interferes with the analysis.

When donating whole blood, in addition to the blood collection on site during the donation, the filling of the sample containers, which are necessary for analysis in the laboratory, usually takes place at the same time. The sample containers contain an anticoagulant so that the proteins in the blood do not precipitate until they are checked in the laboratory. The anticoagulant can be Na-citrate, CPD (citrate-phosphate-dextrose), CP2D (citrate-phosphate-double-dextrose), CPDA (citrate-phosphate-dextrose-adenine) or ACD (“acid-citrate-dextrose”). Sample containers are proposed, for example, in WO 2003/095974 A2.

According to one example, blood is collected for sampling in an enlarged section of the supply line to the whole blood bag. Once the desired volume of blood for sampling has been collected in the enlarged tubing section, the supply line to the whole blood bag is closed between the whole blood bag and the enlarged section, the needle is removed from the donor and the blood from the tube section is transferred to sample containers. Such a blood bag system has proven to be less suitable for the present application.

A blood bag system for umbilical cord blood has been proposed, for example, in DE 202013011482 U1 for the collection of umbilical cord blood. Here, the whole blood bag serves as a freezing bag and the enlarged tube section as a reservoir area.

### OBJECT OF THE INVENTION

Although the known techniques generally work satisfactorily, efforts continue to be made to provide improvements in the field of blood collection and blood product recovery, particularly with regard to blood bag systems that allow a higher degree of automation. The present invention aims to simplify and automate the analysis of whole blood and to simplify and systematize the sampling process.

### SUMMARY OF THE INVENTION

These and other objects are solved by the subject matter of the independent patent claims. Preferred embodiments are the subject of the subclaims or are described below.

The object of the present invention is a predonation bag system and a blood bag system comprising the predonation bag system which enables the removal of blood from a predonation bag as part of the blood bag system into sample containers no longer locally at the site of blood collection, but centrally and preferably automatically in a laboratory during or at the time of blood processing.

The predonation bag system comprises at least one predonation bag, a venipuncture adapter and a blood collection line with
a) a first tube section that can be sealed off from the blood collection line as a feed from the blood collection line into the predonation bag and
b) a second tube section as a feed from the blood collection line into further parts of the blood bag system, e.g. into a whole blood bag (whereby the whole blood bag is not part of the predonation bag system), in particular directly into a whole blood bag,
wherein the predonation bag or the first tubing section or both comprises/comprise an anticoagulant for whole blood and the anticoagulant is EDTA. In particular, the anticoagulant is K2-EDTA (di-potassium EDTA) and/or K3-EDTA (tri-potassium EDTA). The sample container then no longer needs to contain an anticoagulant.

For example, the first tube section and the second tube section branch off from the blood collection line. The second tube section can also be the same line as the blood collection line, which is then called the second tube section after the branching off of the first tube section and, conversely, the first tube section can also be the same line as the blood collection line, which is then called the first tube section after the branching off of the second tube section.

One of the advantages of filling the sample containers centrally is that the process steps can be controlled. A lot of donor information is stored in the blood donation service provider's database, which can be called up so that a decision on the number and type of tests required and therefore the sample containers to be filled can only be made after comparison with the available data. Another advantage is the central, automated sealing of the predonation bags. The risk of the bag becoming aerated is significantly lower than if the bag is sealed or otherwise separated at the blood donation site.

For transportation and subsequent filling of the sample containers, the whole blood in the predonation bag of the blood bag system, from which the sample container(s) will later be filled, is provided with a suitable anticoagulant. This prevents the blood from clotting. Surprisingly, EDTA, in particular K2-EDTA and/or K3-EDTA, has proven to be a suitable anticoagulant for the whole blood in the predonation bag. K2-or K3-EDTA can be used in infection and blood group serology laboratories as well as in PCR analysis and molecular blood grouping. These laboratories perform a large number of tests (e.g. >10), all of which must be switched to the same anticoagulant.

A further characteristic of the blood bag systems and the predonation bag systems as part of the blood bag system, respectively, according to the invention is that the anticoagulant in the whole blood of the predonation bag is different from that in the whole blood bag. This requires that the whole blood in the predonation bag can be collected in such a way that neither the anticoagulant for the predonation bag nor the whole blood in which the anticoagulant is dissolved comes into contact with the donor or donor blood or the whole blood in the whole blood bag; in particular, contact or mixing with the whole blood in the whole blood bag must be avoided. CPD, CPDA1 and/or CP2D can then be used in the whole blood bag as an anticoagulant, for example.

The invention further relates to a method for the collection of whole blood by means of the blood bag system, wherein the whole blood provided with anticoagulant or a part thereof from the predonation bag is filled into one or more sample containers after transportation of the predonation bag to a laboratory, preferably automated, and the sample container is supplied to an analysis or the sample containers are each supplied to different analyses.

The number of sample containers per predonation bag can be determined depending on the donor and a comparison with an entry in a database for the donor. For example, the database can be used to determine which type of analysis is required for the respective donor and how many sample containers are required for this. A predonation bag or blood bag system is assigned to each donor. The automation allows the correct number of sample containers to be selected, unnecessary sample containers to be avoided, and the sample containers to be arranged for the same tests or sequences of the same tests for the different donors. For example, first-time donors are subjected to a larger number of tests and more sample containers are required accordingly. Furthermore, the work step of filling and labeling the sample containers at the blood donation site is avoided.

### DETAILED DESCRIPTION OF THE INVENTION

According to the present invention, the sampling of whole blood is not carried out from the whole blood bag itself, but the flow path of the whole blood is clamped before the whole blood bag and the whole blood taken from the donor is first diverted into the sample bag (predonation bag), e.g. via a T- or Y-branch until the flow path to the predonation bag is closed, in particular by an initially opened and later closed closure in the area of the first tube section, and the flow path to the whole blood bag is opened, in particular by an initially closed and later opened closure in the area of the second tube section, in order to fill the whole blood bag.

According to this embodiment, a disposable tubing set is used as part of the blood bag system, which is placed on the main flow path from the blood collection line into the second tubing section with the first tubing section as a “dead end” and has the predonation bag, a first tubing section and an adapter for the sample container as a sampling point. Blood can then be taken from the predonation bag, for example, by opening a lid on the adapter, inserting a sample container, e.g. in the form of a sample tube, into the adapter and piercing the membrane of the sample container with a needle in the adapter. Whole blood then flows via the sampling point into the attached sample container, which is under negative pressure, for example.

According to one embodiment of the invention, the internal volume of the predonation bag is 10 to 50 mL, in particular 25 to 35 mL, or contains the previously stated volume of whole blood. In particular, the concentration of anticoagulant in the whole blood is 1.2 to 2 mg/mL, preferably 1.4 to 1.8 mg/mL, such as about 1.6 mg/mL. The anticoagulant in the predonation bag ensures that the desired tests can be carried out with the whole blood in the sample container(s) in the laboratory with a time delay. The anticoagulant in the predonation bag used according to the invention does not interfere with the laboratory analysis and thus behaves neutrally in the tests.

The anticoagulant can be present in the predonation bag or in the first tube section, preferably beyond the closure in the first tube section and towards the predonation bag:
1. as a liquid, e.g. dissolved in water,
2. as a coating, i.e. immobilized as part of an interior wall coating,
3. as a solid (e.g. as a tablet), and/or
4. as a solid or liquid in a capsule that opens by external pressure or by dissolving.

The anticoagulant can, for example
a) applied as a solid to the inner surface of the first tube section or of a sample tube inserted into the first tube section, and/or
b) be applied as a solid to the inner surface of the predonation bag.

According to one embodiment of the invention, the anticoagulant is introduced into the predonation bag dissolved in water. This ensures that the EDTA is easily distributed in the whole blood when the whole blood flows into the predonation bag, e.g. due to the pressure in the donor's bloodstream.

The anticoagulant can also be immobilized on the inner wall of the predonation bag and/or in a tube section or on a small tube inserted into the tube section towards the predonation bag after the sealing facility (closure). Contact of the whole blood with the immobilized EDTA causes it to dissolve. The inner walls are coated, e.g. by spray drying.

It is also possible that a suitable quantity of anticoagulant is present in solid form in the predonation bag and that the anticoagulant dissolves in the whole blood as soon as it flows into the predonation bag and comes into contact with it, possibly assisted by shaking the predonation bag.

A tablet, e.g. in a pill chamber, can also be inserted into the tube section (first tube section) towards the predonation bag and dissolves as the whole blood flows past and, if necessary, subsequent shaking (manually or on the mixing scale). The tablet can also simply be placed in the predonation bag.

According to another embodiment, the anticoagulant is introduced into a capsule whose shell dissolves on contact with whole blood or which must be broken open mechanically so that the anticoagulant is released in the predonation bag or in a supply line to it. For example, the capsule is broken if the supply line to the predonation bag is closed behind the location of the capsule. A backflow to the donor or in the direction of the whole blood bag must be prevented.

In particular, the first tube section has a closure (preferably initially open and later sealable). The anticoagulant is preferably arranged exclusively in the first tube section behind the closure and/or in the predonation bag (also behind the closure in the first tube section).

A valve or a closure in the form of a mechanical lock in the supply line to the predonation bag, which can be closed and only opened again if necessary, ensures that the EDTA or whole blood with the EDTA it contains cannot flow back. This effectively prevents the anticoagulant from coming into contact with the donor and/or the whole blood in the whole blood bag.

According to one embodiment, the closure in the first tube section is initially closed in the basic position, can then be opened and closes the second time it is closed, in particular irreversibly, i.e. cannot be opened a second time. Or the closure is initially open in the basic position and closes, in particular irreversibly, when it is first closed and cannot then be opened a second time.

The second tube section can, for example, have a break-off part with a predetermined breaking point inside the second tube section as a closure, which is mechanically broken off and thereby releases the flow into the whole blood bag via the second tube section. Equally, but less preferably, the break-off part can be arranged in the first tube section. An example of a possible design of the break-off part as a closure that can be irreversibly opened can be seen in DE 3030020 A1.

The central sample container filling can be realized in various ways. According to one embodiment, the predonation bag is separated from the rest of the blood bag system by sterile welding (in particular between the closure of the first tube section and the predonation bag), whereby both ends are closed by welding or the supply line can be separated behind the closure when viewed from the predonation bag. Welding is carried out, for example, by thermal heating and compression of the tube section in certain areas and separation, e.g. by punching in the welding zone or forming a tear-off line as material weakening in the welding zone. In this way, both tube ends are sealed by welding.

The sample containers are then filled centrally in the laboratory with blood from the predonation bag. This step can be carried out automatically. For this purpose, the specific preparation number or identification of the predonation bag is first read out and the desired number of sample containers is determined, whereby the sample containers can always be clearly assigned to the individual donation, e.g. by providing each sample container with an identification that allows it to be assigned to the donor. This can be done by labeling or by assigning a sample identification and/or a donor identification in a database. The whole blood mixed with EDTA is then transferred from the predonation bag to the sample container. One option for filling the sample container is to use an adapter, as described above, which can also be used for manual sample container filling. However, it is also possible to fill the container directly using a tube or cannula. The cannula can be used to puncture the predonation bag in order to take a sample. Contamination with foreign blood must be avoided.

If desired, the predonation bag system can also be welded off from the rest of the blood bag system, in particular between the clamp on the second tube section and the whole blood bag, by sealing the second tube section on both sides by sterile welding (e.g. as described above) and creating a separation in the welded area, e.g. by punching, or by pre-stamping a tear-off line to weaken the material.

The invention is explained with reference to the following figures, without being limited to them. It shows:

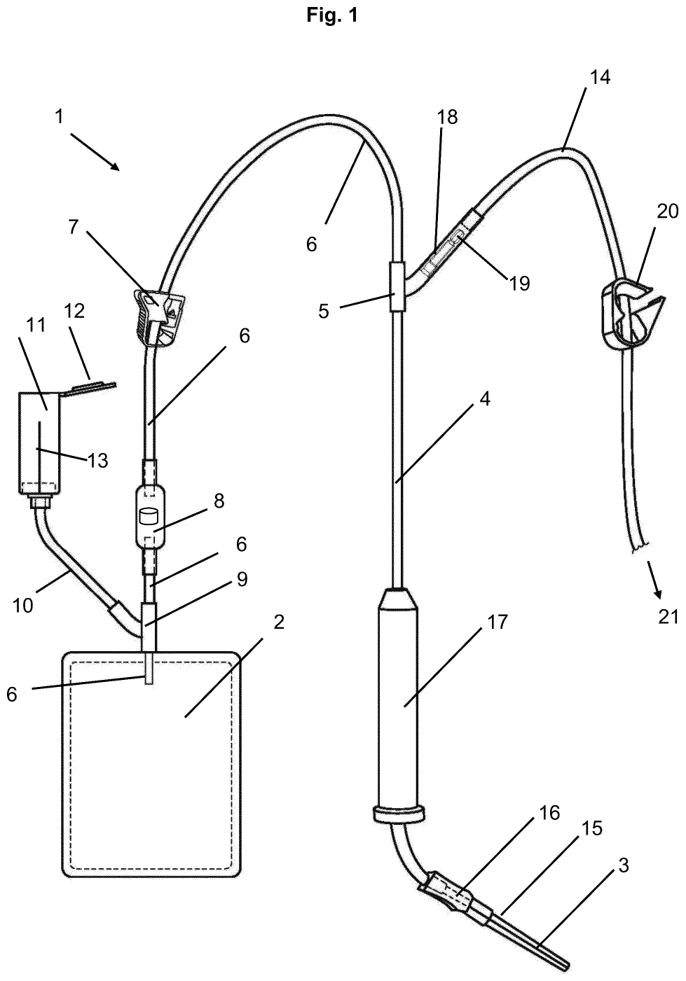

FIG. 1 the predonation bag system and

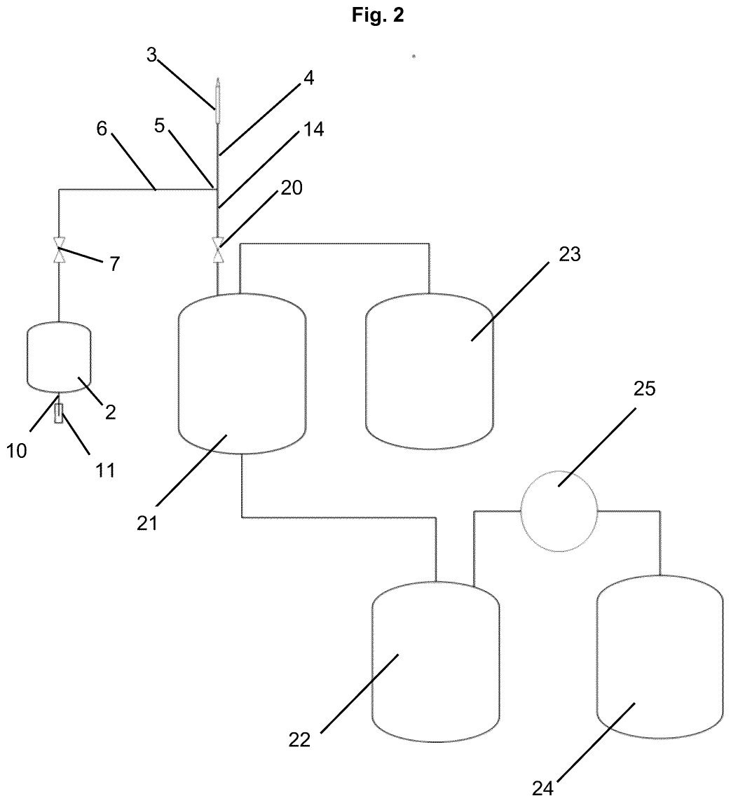

FIG. 2 a blood bag system with predonation bag system.

FIG. 1 shows an illustration of the predonation bag system 1. The predonation bag system 1 comprises a predonation bag 2, a venipuncture needle 3, a blood collection tube 4 from the venipuncture needle 3 to a first Y-connection 5, from which the flow from the blood collection tube 4 continues into a first tube section 6, which ends in the predonation bag 2.

The other arm of the Y-connection 5 leads into a second tube section 14 which leads to the whole blood bag 21.

The venipuncture needle 3 is connected to the free end of the blood collection line 4 via a venipuncture adapter 16 and is used to collect whole blood from the donor. The venipuncture needle 3 is enclosed by an attachable tapered cap 15. Furthermore, a protective bell 17 can be moved along the blood collection tube 4 and engages on the adapter at the end of the blood collection tube so that the protective bell 16 protectively surrounds the venipuncture needle 3 and the cap 15 and holds them inside.

The second tubesection 14 branches off from the blood collection line 4 at the other arm of the Y-connection 5. Part of the Y-connection 5 is a break-off part 18 inserted into this arm of the Y-connection, which blocks the flow through the arm of the Y-connection 5. The break-off part 18 has a hollow sleeve and an olive 19 at the end, which closes the sleeve in the direction of flow. The sleeve fits tightly into the arm of the Y-connection at the other end with a collar. By bending the end of the arm, the olive can be broken off along a predetermined breaking point, releasing the flow of whole blood into the second tube section 14 through the sleeve.

The olive 19 remains at the end of the arm of the Y-piece because its outer circumference has ridges which prevent the olive 19 from being swept into the second tube section 14, but on the other hand allow the blood to pass through, for example, comb grooves in the olive. The break-off part 18 is an option and can also be inserted in the same way into the first tube section 6 or the arm of the Y-connection 5, which leads into the first tube section 6.

In addition, the second tube section 14 is provided with an initially open and later reclosable tube clamp 20, which is closed after the whole blood bag 21 (see FIG. 2) is sufficiently filled and is intended to prevent whole blood from flowing back.

EDTA in the form of a tablet is introduced into the pill chamber 8 in the first tube section 6. By flowing past, the tablet dissolves and releases the EDTA into the whole blood, so that the whole blood in the predonation bag 2 is ultimately stabilized by means of the anticoagulant EDTA.

Via a second Y-connection 9, a sampling adapter 11 is in flow connection with the predonation bag 2 via a sampling tube section 10 over a section of the first tube section. If the lid 12 of the sampling adapter 11 is opened, a sample container with the septum can be inserted into the cylindrical cavity of the sampling adapter 11. Due to the negative pressure in the sample container, the sample container fills with the whole blood containing the anticoagulant. In the same way, further sample containers, e.g. in the form of sampling tubes, can be automatically labeled and filled in a laboratory, i.e. not at the site of blood collection. These are then collected and fed to the corresponding test in a plurality. The arms of the Y-connections are each part of the corresponding tube sections.

FIG. 2 schematically shows a blood bag system comprising the predonation bag system 1 of FIG. 1 (however, here the sampling adapter 11 is connected directly to the predonation bag 2 via the sampling tube section 10 without a second Y-connection).

Via a venipuncture needle 3, the donor's whole blood is directed into the blood bag system during the blood collection, whereby a first tube section 6 after the Y-connection 5 directs the donor's whole blood to the predonation bag 2 and a second tube section 14 to the whole blood bag 21. First, the whole blood collected via the venipuncture needle 3 is passed through the blood collection tube 4 and the Y-connection 5 into the first tube section 6 via an irreversible clamp 7 into the predonation bag 2. “Irreversible clamp” 7 means that it is either open in the basic state (preferred) or closed in the basic state and can be opened, but for both constellations it can no longer be opened after the first closing. The predonation bag 2 is connected to an adapter 11 via a sampling tube section 10, which is intended for filling sample containers. The adapter 11 can be opened, exposing a cylindrical guide, and has a needle inside it for piercing a septum. As soon as the predonation bag 2 is sufficiently filled, the irreversible clamp 7 is closed and the reversible clamp 20 in the second tube section 14 is opened or the olive 19 is also broken off. Now the whole blood flows exclusively into the whole blood bag 21, which is often also called the collection bag. It is also possible that only the initially closed clamp 20 or only the break-off part 18 are arranged in or on the second tube section.

When processing into individual preparations, the whole blood in the whole blood bag 21 is first centrifuged. The components are then separated: Erythrocytes in the erythrocyte bag 22, plasma in the plasma bag 23 and thrombocytes (remain in the whole blood bag 21) are separated. In Germany, only leukocyte-depleted blood products or products with a very low leukocyte content may be marketed in accordance with the specifications of the federal authority, the Paul Ehrlich Institute. Leukocyte depletion means the removal of leukocytes from the preparations. As a rule, the white blood cells are removed from the erythrocyte concentrate using a filter 25, as shown in FIG. 2, so that the leukocyte content per unit of preparation after filtration is less than 10⁻⁶ leukocytes in the bag 24 for the leukocyte-depleted erythrocyte concentrate. Surface-modified polyester fibers can be used as filter media. Filtration takes place by adsorption of the leukocytes. The bag 24 for the leukocyte-depleted erythrocyte concentrate is provided with an additive solution.

1. Blood bag system comprising a predonation bag system and at least one whole blood bag, wherein the predonation bag system comprises at least:
a predonation bag, a venipuncture adapter and a blood collection line with
a) a first tube section which is sealable with respect to the blood collection line as a feed from the blood collection line into the predonation bag and
b) a second tube section as a feed from the blood collection line into the whole blood bag,
wherein the predonation bag or the sealable first tube section or both comprises/comprise a first anticoagulant for whole blood and the first anticoagulant is EDTA; wherein the predonation bag or the sealable first tube section comprises an adapter for filling of one or more sample containers; and
wherein in the whole blood bag a second anticoagulant is contained which is different from the first anticoagulant and which is not EDTA and is CPD, CPDA1 and/or CP2D.

2. Blood bag system according to claim 1, wherein the first anticoagulant is K2-EDTA or K3-EDTA or both.

3. Blood bag system according to claim 1, wherein the first anticoagulant
a) is applied as a solid to the inner surface of the sealable first tube section or of a small tube inserted into the sealable first tube section and/or
b) is applied as a solid to the inner surface of the predonation bag.

4. Blood bag system according to claim 1, wherein the first anticoagulant is introduced into the predonation bag dissolved in water.

5. Blood bag system according to claim 1, wherein the predonation bag has a volume of 10 to 50 mL, in particular 25 to 35 mL.

6. Blood bag system according to claim 1, wherein the first anticoagulant is introduced in an amount such that a concentration in the whole blood of the predonation bag of greater than or equal to 1.2 mg/mL, and preferably 1.4 to 1.8 mg/mL is obtained.

7. Blood bag system according to claim 1, wherein the first anticoagulant is introduced as a pill or tablet into the predonation bag or the sealable first tube section, in particular in a pill chamber.

8. Blood bag system according to claim 1, wherein the sealable first tube section and the predonation bag are formed as a dead end.

9. Blood bag system according to claim 1, wherein the sealable first tube section has an initially open and later sealable closure, in particular later irreversibly closeable.

10. Blood bag system according to claim 1, wherein the first tube section has a break-off part with a predetermined breaking point inside the first tube section, which is mechanically broken off and thereby releases the flow into the predonation bag via the sealable first tube section and/or
wherein the second tube section has a break-off part with a predetermined breaking point inside the second tube section, which is mechanically broken off and thereby releases the flow into the whole blood bag via the first tube section.

11. Blood bag system according to claim 1, wherein the sealable first tube section comprises a sealable closure, wherein the first anticoagulant is preferably arranged exclusively in the sealable first tube section behind the closure and/or exclusively in the predonation bag.

12. Blood bag system according to claim 1, wherein a reversible clamp is located in the second tube section for opening and closing again and, optionally, reopening the inflow into the whole blood bag.

13. Blood bag system according to claim 1, wherein a clamp is located in the second tube section, which is initially closed, can be opened and then closes again reversibly or irreversibly.

14. Method for collecting whole blood by means of a blood bag system according to claim 1, wherein the whole blood provided with first anticoagulant or a part thereof from the predonation bag after transportation to a laboratory is filled by means of the adapter into one or more sample containers, preferably automatically, and the sample container is supplied to an analysis or the sample containers are each supplied to different analyses.

15. Method according to claim 14, wherein the number of sample containers per predonation bag is determined as a function of the donor and a comparison with an entry in a database for the donor and the sample containers and their number are labeled with regard to the tests selected by means of the entry in the database and preferably sample containers of different donors, wherein only one defined blood bag system is assigned to each donor, are automatically sorted on the basis of the labeling and are compiled with regard to identical tests or sequences of identical tests for the different donors.
